(19) United States
(12) Patent Application Publication
(10) Pub. No.: US 2022/0291224 A1
(43) Pub. Date: Sep. 15, 2022

(54) COLORIMETRIC BIOSENSOR, DISCOLORATION KIT, AND SENSING METHOD USING SAME

(71) Applicant: JEN-LIFE, Busan (KR)

(72) Inventors: Jin-Woo OH, Busan (KR); Eun Jeung CHOI, Busan (KR); Jong Min LEE, Chuncheon-si, Gangwon-do (KR); Kyounga LIM, Busan (KR); Dong Wook HAN, Busan (KR)

(73) Assignee: JEN-LIFE, Busan (KR)

(21) Appl. No.: 17/635,880

(22) PCT Filed: Dec. 29, 2020
(86) PCT No.: PCT/KR2020/019329

§ 371 Date: Feb. 16, 2022

(30) Foreign Application Priority Data
Dec. 31, 2019 (KR) 10-2019-0178796
Dec. 31, 2019 (KR) 10-2019-0178800
Dec. 31, 2019 (KR) 10-2019-0178802

(51) Int. Cl. G01N 33/66; G01N 31/22; G01N 33/52

(52) U.S. Cl. CPC G01N 33/66; G01N 31/22; G01N 33/52

(57) ABSTRACT

A colorimetric biosensor according to an embodiment includes a transition metal, a solvent and an additive. The colorimetric biosensor further includes M13 bacteriophage. The colorimetric biosensor is for sensing glucose, lactate or pyruvate. A colorimetric biokit according to an embodiment, includes a plurality of arrays, each having a colorimetric biosensor including a transition metal, a solvent, and an additive. Colors of the arrays change depending on the presence or absence of an analyte. The analyte is glucose, lactate, or pyruvate.

### CROSS-REFERENCE TO PRIOR APPLICATIONS

This application is a National Stage Patent Application of PCT International Patent Application No. PCT/KR2020/019329 (filed on Dec. 29, 2020) under 35 U.S.C. § 371, which claims priority to Korean Patent Application Nos. 10-2019-0178802 (filed on Dec. 31, 2019), 10-2019-0178800 (filed on Dec. 31, 2019), and 10-2019-0178796 (filed on Dec. 31, 2019), which are all hereby incorporated by reference in their entirety.

### BACKGROUND

The present invention relates to a colorimetric biosensor, a colorimetric biokit, and a sensing method using the same, and more specifically, to a colorimetric biosensor or a colorimetric biokit on which small arrays, each comprising, as main components, a transition metal, a solvent, and bacteriophage are mounted, and in which the colors of the small arrays change depending on the presence or absence of an analyte, and a sensing method using the same.

Diabetes mellitus, one of the most widely known diseases in the modern world, is a metabolic disease characterized by hyperglycemia caused by insufficient secretion or dysfunction of insulin which is necessary for blood glucose control in the body. Chronic hyperglycemia causes damage and dysfunction of each organ in the body, which cause microvascular complications and macrovascular complications, thereby increasing mortality. Diabetes is diagnosed by measuring blood sugar levels, that is, glucose levels, and at this time, blood is usually sampled and tested by a glucose sensor. A commonly used enzyme-based glucose biosensor is a disposable biosensor that is difficult to reuse, because it has a limited lifetime due to the instability of the enzyme and is difficult to store and the process of purifying the enzyme is complicated. In addition, an enzyme-free glucose sensor has insufficient selectivity and is intended to come into contact with a sample, and thus when it is used on the human body, an invasive process is performed.

Lactate and pyruvate are known to be fatigue substances that are produced in the body after exercise. For this reason, lactate and pyruvate sensors are widely used in the field to understand the training effect and training state, and there is a need for biosensors capable of simply and easily detecting and measuring lactate and pyruvate.

In response to this need, a lactic acid sensor including a phosphate for improving the activity of lactic acid oxidase in a reaction layer was proposed (Japanese Patent No. 3498105). In addition, examples of methods for fabricating biosensors such as pyruvate sensors include a method including a step of forming an inorganic gel layer, which comprises at least a mediator, a surfactant, a buffer and a layered inorganic compound, on the upper surface of a substrate having an electrode (Japanese Patent No. 4088312).

However, these lactate and pyruvate-related sensors have problems in that they are expensive, quantitative analysis using these sensors is difficult, and a lot of time is required to detect the presence or absence of the analyte.

Therefore, the present inventors have made efforts to overcome the above-described problems, and as a result, have recognized that it is urgent to develop a colorimetric biosensor, a colorimetric sensor in which the colors of small arrays change depending on the presence or absence of analyte, and a sensing method using the same, thereby completing the present invention.

### SUMMARY

An object of the present invention is to provide a colorimetric biosensor, a colorimetric biokit on which small arrays, each comprising, as main components, a transition metal, a solvent, and bacteriophage, are mounted, and in which the colors of the small arrays change depending on the presence or absence of an analyte, and a sensing method using the same.

Another object of the present invention is to provide a colorimetric biosensor, a colorimetric biokit comprising the same, and a sensing method using the same, which have a maximized color contrast effect resulting from an increased color change amount due to an improved property of changing color in the presence of an analyte.

Objects of the present invention are not limited to the above-mentioned objects, and other objects not mentioned herein may be clearly understood by those of ordinary skill in the art from the following description of the invention.

To achieve the above objects, the present invention provides a colorimetric biosensor, a colorimetric biokit, and a sensing method using the same.

Hereinafter, the present specification will be described in more detail.

The present invention provides a colorimetric biosensor comprising a transition metal, a solvent and an additive.

In the present invention, the colorimetric biosensor may further comprise M13 bacteriophage.

In the present invention, the colorimetric biosensor may be for sensing glucose, lactate or pyruvate.

In the present invention, the transition metal may be at least one selected from the group consisting of copper (Cu), iron (Fe), cobalt (Co) and vanadium (V).

In the present invention, the solvent may be at least one selected from the group consisting of water, acetone, methanol, ethanol, n-propanol, isopropanol, n-butanol, chloroform, dichloromethane (DCM), toluene, tetrahydrofuran (THF), pyridine, diethyl ether, ethyl acetate, nitromethane, acetonitrile, ammonia, N,N-dimethylformamide (DMF), dimethyl sulfoxide (DMSO), formic acid, and propylenecarbonate.

In the present invention, the additive may be at least one selected from the group consisting of a halogen, sodium fluoride (NaF), sodium cyanate (NaNCO), sulfuric acid (H₂SO₄), nitric acid (HNO₃), oxalic acid, hydrogen peroxide (H₂O₂), hydrogen cyanide (HCN), sodium hydroxide (NaOH), hydroxylamine, ethylene amine, 4-nitrophenol, dithionate (S₂O₆²⁻), diborane, sodium borohydride, iodine (I₂), tris-2 carboxyethyl phosphine hydrochloride (TCEP), and dimethylamine borane (DMAB).

In addition, the present invention provides a colorimetric biokit comprising a plurality of arrays, each consisting of a colorimetric biosensor comprising a transition metal, a solvent, and an additive.

In the colorimetric biokit of the present invention, the colors of the arrays may change depending on the presence or absence of an analyte.

In the present invention, the analyte may be glucose, lactate, or pyruvate.

In the present invention, when the analyte is glucose, the colorimetric biokit may have the configuration shown in Table 1 below.

TABLE 1

| 1 | 2 | 3 |
|---|---|---|
| Copper-acetone- | Copper-water- | Copper-ethanol- |
| hydroxylamine | hydroxylamine | hydroxylamine |
| 4 | 5 | 6 |
| Copper-propylene | Iron-isopropanol- | Copper-methanol- |
| carbonate-hydroxylamine | hydroxylamine | hydroxylamine |
| 7 | 8 | 9 |
| Copper-DMSO- | Copper-DMF- | Cobalt-pyridine- |
| hydroxylamine | hydroxylamine | hydroxylamine |

In the present invention, when the analyte is glucose, it may be determined that glucose is present, when the root mean square (RMS) for ΔRGB, quantified based on the RGB coordinates of each array consisting of the colorimetric biosensor that changed color by addition of glucose, and the initial RGB coordinates of each array consisting of the colorimetric biosensor to which glucose has not been added, is 20 to 300.

In the present invention, when the analyte is lactate, the colorimetric biokit may have the configuration shown in Table 2 below.

TABLE 2

| 1 | 2 | 3 |
|---|---|---|
| Copper-4-nitrophenol- | Copper-4-nitrophenol- | Copper-4-nitrophenol- |
| ethanol | isopropanol | methanol |
| 4 | 5 | 6 |
| Copper-DMAB-water | Copper-4-nitrophenol- | Copper-iodine- |
| | water | acetonitrile |
| 7 | 8 | 9 |
| Vanadium-halogen | Copper-DMAB- | Copper-DMAB- |
| (Cl)-acetone | acetonitrile | acetone |

In the present invention, when the analyte is lactate, it may be determined that lactate is present, when the root mean square (RMS) for ΔRGB, quantified based on the RGB coordinates of each array consisting of the colorimetric biosensor that changed color by addition of lactate, and the initial RGB coordinates of each array consisting of the colorimetric biosensor to which lactate has not been added, is 20 to 250.

In the present invention, when the analyte is pyruvate, the colorimetric biokit may have the configuration shown in Table 3 below.

TABLE 3

| 1 | 2 | 3 |
|---|---|---|
| Copper- | Copper- | Copper- |
| hydroxylamine- | hydroxylamine- | hydroxylamine- |
| acetone | pyridine | acetonitrile |
| 4 | 5 | 6 |
| Copper- | Copper- | Vanadium-hydrogen |
| hydroxylamine-water | hydroxylamine-ethanol | peroxide-DMF |
| 7 | 8 | 9 |
| Vanadium-halogen | Vanadium-hydrogen | Vanadium-hydrogen |
| (Cl)-DMF | peroxide-ethanol | peroxide-isopropanol |

In the present invention, when the analyte is pyruvate, it may be determined that pyruvate is present, when the root mean square (RMS) for ΔRGB, quantified based on the RGB coordinates of each array consisting of the colorimetric biosensor that changed color by addition of pyruvate, and the initial RGB coordinates of each array consisting of the colorimetric biosensor to which pyruvate has not been added, is 20 to 300.

The present invention also provides a sensing method using a colorimetric biosensor comprising steps of:

(S1) dropping an analyte into a colorimetric biokit comprising a plurality of arrays; and

(S2) sensing color changes of the plurality of arrays having the analyte dropped thereinto.

All details mentioned in the colorimetric biosensor, the colorimetric biokit, and the sensing method using the same are equally applied unless they are contradictory.

According to the colorimetric biosensor, the colorimetric biokit mounted with small arrays, each comprising, as main components, a transition metal, a solvent, and bacteriophage, and the sensing method using the same according to the present invention, the colors of the small arrays may change depending on the presence or absence of an analyte, and this color change may be confirmed visually.

In addition, the colorimetric biosensor, the colorimetric biokit, and the sensing method using the same according to the present invention may have a maximized color contrast effect resulting from an increased color change amount due to an improved property of changing color in the presence of an analyte, and thus may have significantly improved selectivity, sensitivity, reproducibility, reaction rate, etc.

Effects of the present invention are not limited to the above-mentioned effects, and other effects not mentioned herein will be clearly understood by those skilled in the art from the following description and the appended claims.

### BRIEF DESCRIPTION OF THE DRAWINGS

FIG. 1 depicts images showing the results of sensing glucose using a 3×3 glucose colorimetric kit comprising arrays, each consisting of a glucose colorimetric sensor according to the present invention.

FIG. 2 depicts images showing the results of sensing lactate using a 3×3 lactate colorimetric kit comprising arrays, each consisting of a lactate colorimetric sensor according to the present invention.

FIG. 3 depicts images showing the results of sensing pyruvate using a 3×3 pyruvate colorimetric kit comprising arrays, each consisting of a pyruvate colorimetric sensor according to the present invention.

### DETAILED DESCRIPTION

The terms used in the present specification are currently widely used general terms selected in consideration of their functions in the present invention, but they may change depending on the intents of those skilled in the art, precedents, or the advents of new technology. Additionally, in certain cases, there may be terms arbitrarily selected by the applicant, and in this case, their meanings are described in a corresponding description part of the invention. Accordingly, the terms used in the present invention should be defined based on the meaning of the term and the entire contents of the present invention, rather than the simple term name.

Unless otherwise defined, all terms used herein, including technical and scientific terms, have the same meaning as commonly understood by one of ordinary skill in the art to which the present invention pertains. The terms used in general and defined in dictionaries should be interpreted as having meanings identical to those specified in the context of related technology. Unless definitely defined in the present application, the terms should not be interpreted as having ideal or excessively formative meanings.

A numerical range includes numerical values defined in the range. Every maximum numerical limitation given throughout the present specification includes every lower numerical limitation, as if such lower numerical limitations were expressly written herein. Every minimum numerical limitation given throughout the present specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Hereinafter, embodiments of the present invention will be described in detail, but it is obvious that the present invention is not limited by the following embodiments.

Colorimetric Biosensor

The present invention provides a colorimetric biosensor comprising a transition metal, a solvent and an additive, which may change color depending on the presence or absence of an analyte.

In addition, the colorimetric biosensor may further comprise M13 bacteriophage.

The M13 bacteriophage is a particle having a length of 880 nm and a width of 6.6 nm, and is a protein expressed from a certain gene, unlike nanoparticles produced through general organic or inorganic synthesis, and all the particles have a perfectly identical shape. Thus, there is a great advantage in the preparation of the material. In addition, the M13 bacteriophage is a nanoparticle having a high surface-to-volume ratio, and has about 2,700 pairs of proteins (pVIII proteins) on the surface thereof and 4 to 5 pairs of protein (pIII, pVI, pVII, pIX) at both ends thereof. In particular, in the case of pVIII proteins expressing 2,700 pairs of identical peptides, protein molecules paired at intervals of about 3.3 nm are arranged very densely in a spiral shape. Genes in the bacteriophage may be appropriately recombined, and thus a desired type of peptide can be expressed from each corresponding surface protein. The bacteriophage is easy to apply to various biosensing fields according to the intended use.

The transition metal may be at least one selected from the group consisting of copper (Cu), iron (Fe), cobalt (Co), chromium (Cr), manganese (Mn), nickel (Ni), and vanadium (V). Preferably, it may be at least one selected from the group consisting of copper, iron, cobalt and vanadium.

The term “transition metal” collectively refers to the elements of periods 4 to 7 and groups 3 to 12 of the periodic table. In the case of elements other than the transition metal, the chemical properties of the main group elements in one period change greatly as the number of valence electrons changes, but the transition metals show many similarities not only in a given group but also in the same period. In addition, the transition metal has luster, electrical conductivity, and thermal conductivity, like general metals, and particularly, can form an ionic compound with a non-metal. When the transition metal forms an ionic compound with a non-metal, the transition metal may exist in the form of a complex ion together with a certain number of ligands, unlike a general metal. The complex ion refers to an ion produced by bonding between the transition metal and a ligand.

The solvent may be at least one selected from the group consisting of water, acetone, methanol, ethanol, n-propanol, isopropanol, n-butanol, chloroform, dichloromethane (DCM), toluene, tetrahydrofuran (THF), pyridine, diethyl ether, ethyl acetate, nitromethane, acetonitrile, ammonia, N,N-dimethylformamide (DMF), dimethyl sulfoxide (DMSO), formic acid, and propylenecarbonate. Preferably, the solvent may be at least one selected from the group consisting of water, acetone, methanol, ethanol, isopropanol, pyridine, acetonitrile, N-dimethylformamide, dimethyl sulfoxide, and propylene carbonate.

The additive may be at least one selected from the group consisting of a halogen, sodium fluoride (NaF), sodium cyanate (NaNCO), sulfuric acid (H₂SO₄), nitric acid (HNO₃), oxalic acid, hydrogen peroxide (H₂O₂), hydrogen cyanide (HCN), sodium hydroxide (NaOH), hydroxylamine, ethylene amine, 4-nitrophenol, dithionate (S₂O₆²⁻), diborane, sodium borohydride, iodine (I₂), tris-2 carboxyethyl phosphine hydrochloride (TCEP), and dimethylamine borane (DMAB). Preferably, the additive may be at least one selected from the group consisting of a halogen, hydrogen peroxide, hydroxylamine, 4-nitrophenol, iodine, and dimethylamine borane.

The colorimetric biosensor may comprise the transition metal, the solvent and the additive at a concentration ratio of 1:10 to 25:0.5 to 1.2.

The colorimetric biosensor may be for sensing glucose, lactate or pyruvate.

Colorimetric Biokit

The present invention provides a colorimetric biokit comprising a plurality of arrays, each consisting of a colorimetric biosensor comprising a transition metal, a solvent and an additive.

The colorimetric biosensor is the same as mentioned above.

The individual color of each array in the colorimetric biokit may change depending on the presence or absence of an analyte.

The analyte may be glucose, lactate or pyruvate.

When the analyte is glucose, the colorimetric biokit may have the configuration shown in Table 1 below.

TABLE 1

| 1 | 2 | 3 |
|---|---|---|
| Copper-acetone- | Copper-water- | Copper-ethanol- |
| hydroxylamine | hydroxylamine | hydroxylamine |
| 4 | 5 | 6 |
| Copper-propylene | Iron-isopropanol- | Copper-methanol- |
| carbonate-hydroxylamine | hydroxylamine | hydroxylamine |
| 7 | 8 | 9 |
| Copper-DMSO- | Copper-DMF- | Cobalt-pyridine- |
| hydroxylamine | hydroxylamine | hydroxylamine |

When the analyte is glucose, it may be quantitatively determined that glucose is present, when the absolute value of ΔR, obtained from the RGB coordinates of each array consisting of the colorimetric biosensor that changed color by addition of glucose, and the initial RGB coordinates of the color of each array consisting of the colorimetric biosensor to which glucose has not been added, is 20 or more, or when the absolute value of AB is 50 or more, obtained from the RGB coordinates and the initial RGB coordinates, or 50 or more, or when the absolute value of AB is 50 or more while the absolute value of ΔR is 20 or more. In addition, it may be quantitatively determined that glucose is present, when the root mean square (RMS) for ΔRGB, quantified based on the RGB coordinates of each array consisting of the colorimetric biosensor that changed color by addition of glucose, and the initial RGB coordinates of each array consisting of the colorimetric biosensor to which glucose has not been added, is 20 to 300. Preferably, the RMS may be 25 to 250, most preferably 25 to 200.

When the analyte is lactate, the colorimetric biokit may have the configuration shown in Table 2 below.

TABLE 2

| 1 | 2 | 3 |
|---|---|---|
| Copper-4-nitrophenol- | Copper-4-nitrophenol- | Copper-4-nitrophenol- |
| ethanol | isopropanol | methanol |
| 4 | 5 | 6 |
| Copper-DMAB-water | Copper-4-nitrophenol- | Copper-iodine- |
| | water | acetonitrile |
| 7 | 8 | 9 |
| Vanadium-halogen | Copper-DMAB- | Copper-DMAB- |
| (Cl)-acetone | acetonitrile | acetone |

When the analyte is lactate, the individual color of each array in the colorimetric biokit may change depending on the presence or absence of lactate. More specifically, it may be quantitatively determined that lactate is present, when the absolute value of ΔR, obtained from the RGB coordinates of each array consisting of the colorimetric biosensor that changed color by addition of lactate, and the initial RGB coordinates of the color of each array consisting of the colorimetric biosensor to which lactate has not been added, is 20 or more. In addition, it may be quantitatively determined that lactate is present, when the root mean square (RMS) for ΔRGB, quantified based on the RGB coordinates of each array consisting of the colorimetric biosensor that changed color by addition of lactate, and the initial RGB coordinates of each array consisting of the colorimetric biosensor to which lactate has not been added, is 20 to 250. Preferably, the RMS may be 20 to 200, most preferably 20 to 150.

When the analyte is pyruvate, the colorimetric biokit may have the configuration shown in Table 3 below.

TABLE 3

| 1 | 2 | 3 |
|---|---|---|
| Copper- | Copper- | Copper- |
| hydroxylamine- | hydroxylamine- | hydroxylamine- |
| acetone | pyridine | acetonitrile |
| 4 | 5 | 6 |
| Copper- | Copper- | Vanadium-hydrogen |
| hydroxylamine-water | hydroxylamine-ethanol | peroxide-DMF |
| 7 | 8 | 9 |
| Vanadium-halogen | Vanadium-hydrogen | Vanadium-hydrogen |
| (Cl)-DMF | peroxide-ethanol | peroxide-isopropanol |

When the analyte is pyruvate, the individual color of each array in the colorimetric biokit may change depending on the presence or absence of pyruvate. More specifically, it may be quantitatively determined that pyruvate is present, when the absolute value of ΔG, obtained from the RGB coordinates of each array consisting of the colorimetric biosensor that changed color by addition of pyruvate, and the initial RGB coordinates of the color of each array consisting of the colorimetric biosensor to which pyruvate has not been added, is 20 or more, or when the absolute value of ΔB, obtained from the RGB coordinates and the from the initial RGB coordinates, is 15 or more, or when the absolute value of ΔB is 15 or more while the absolute value of ΔG is 20 or more. In addition, it may be quantitatively determined that pyruvate is present, when the root mean square (RMS) for ΔRGB, quantified based on the RGB coordinates of each array consisting of the colorimetric biosensor that changed color by addition of pyruvate, and the initial RGB coordinates of each array consisting of the colorimetric biosensor to which pyruvate has not been added, is 50 to 300. Preferably, the RMS may be 50 to 250, most preferably 50 to 200.

The RMS may be the root mean square of ΔR²+ΔG²+ΔB² based on ARGB obtained from the RGB coordinates of each array consisting of the colorimetric biosensor.

Biosensing Method

The present invention also provides a sensing method comprising steps of:

(S1) dropping an analyte into a colorimetric biokit comprising a plurality of arrays; and

(S2) sensing color changes of the plurality of arrays having the analyte dropped thereinto.

The colorimetric biosensor and the colorimetric biokit are the same as mentioned above.

The colorimetric biosensor, the colorimetric biokit, and the sensing method using the same according to the present invention may have a maximized color contrast effect resulting from an increased color change amount due to an improved property of changing color in the presence of an analyte, and thus may have significantly improved selectivity, sensitivity, reproducibility, reaction rate, etc.

The advantages and features of the present invention, and the way of attaining them, will become apparent with reference to the examples described in detail below. However, the present invention is not limited to the examples disclosed below and may be embodied in a variety of different forms; rather, these examples are provided so that this disclosure of the present invention will be thorough and complete, and will fully convey the scope of the present invention to those skilled in the art. The scope of the present invention will be defined by the appended claims.

### Example 1. Colorimetric Kit Comprising Glucose Colorimetric Sensor

A 3×3 glucose colorimetric kit comprising arrays, each consisting of the glucose colorimetric sensor according to the present invention, was produced. The glucose colorimetric sensor constituting each of the arrays was produced by mixing a transition metal, a solvent and an additive together at a concentration ratio of 1 mM:18.2 mM:0.8 mM, and configurations for the transition metal, the solvent and the solvent are shown in Table 1 below.

TABLE 1

| 1 | 2 | 3 |
|---|---|---|
| Copper-acetone- | Copper-water- | Copper-ethanol- |
| hydroxylamine | hydroxylamine | hydroxylamine |
| 4 | 5 | 6 |
| Copper-propylene | Iron-isopropanol- | Copper-methanol- |
| carbonate-hydroxylamine | hydroxylamine | hydroxylamine |
| 7 | 8 | 9 |
| Copper-DMSO- | Copper-DMF- | Cobalt-pyridine- |
| hydroxylamine | hydroxylamine | hydroxylamine |

### Example 2. Colorimetric Kit Comprising Lactate Colorimetric Sensor

A 3×3 lactate colorimetric kit comprising arrays, each consisting of the lactate colorimetric sensor according to the present invention, was produced. The lactate colorimetric sensor constituting each of the arrays was produced by mixing a transition metal, a solvent and an additive together at a concentration ratio of 1 mM:18.2 mM:0.8 mM, and configurations for the transition metal, the solvent and the solvent are shown in Table 2 below.

TABLE 2

| 1 | 2 | 3 |
|---|---|---|
| Copper-4-nitrophenol- | Copper-4-nitrophenol- | Copper-4-nitrophenol- |
| ethanol | isopropanol | methanol |
| 4 | 5 | 6 |
| Copper-DM AB-water | Copper-4-nitrophenol- | Copper-iodine- |
| | water | acetonitrile |
| 7 | 8 | 9 |
| Vanadium-halogen | Copper-DMAB- | Copper-DMAB- |
| (Cl)-acetone | acetonitrile | acetone |

### Example 3. Colorimetric Kit Comprising Pyruvate Colorimetric Sensor

A 3×3 pyruvate colorimetric kit comprising arrays, each consisting of the lactate colorimetric sensor according to the present invention, was produced. The pyruvate colorimetric sensor constituting each of the arrays was produced by mixing a transition metal, a solvent and an additive together at a concentration ratio of 1 mM:0.8 mM:18.2 mM, and configurations for the transition metal, the solvent and the solvent are shown in Table 2 below.

TABLE 3

| 1 | 2 | 3 |
|---|---|---|
| Copper- | Copper- | Copper- |
| hydroxylamine- | hydroxylamine- | hydroxylamine- |
| acetone | pyridine | acetonitrile |
| 4 | 5 | 6 |
| Copper- | Copper- | Vanadium-hydrogen |
| hydroxylamine-water | hydroxylamine-ethanol | peroxide-DMF |
| 7 | 8 | 9 |
| Vanadium-halogen | Vanadium-hydrogen | Vanadium-hydrogen |
| (Cl)-DMF | peroxide-ethanol | peroxide-isopropanol |

### Experimental Example 1. Glucose Sensing

1.1. Confirmation of Color Change

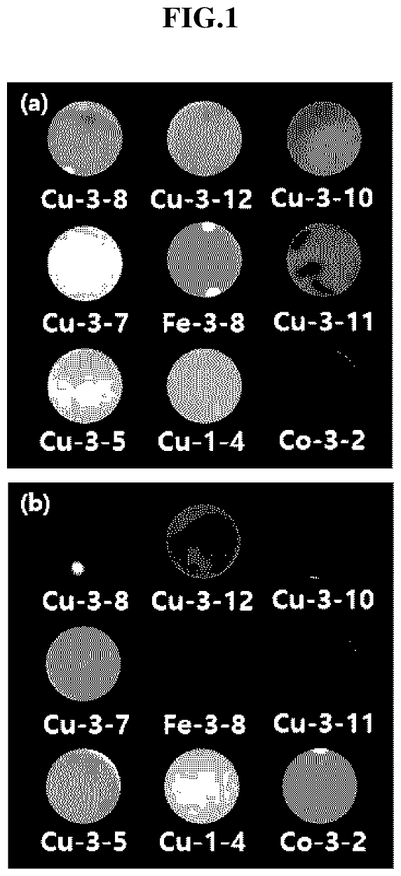

In order to sense and confirm the color change of the 3×3 glucose colorimetric kit produced in Example 1 according to the present invention and comprising arrays, each consisting of the glucose colorimetric sensor, 0.1 M glucose was dropped into the 3×3 glucose colorimetric kit (Example 1). The results are shown in FIG. 1.

Referring to FIG. 1, it could be confirmed that there was a clear difference between the 3×3 glucose colorimetric kit (Example 1) into which glucose was not dropped (FIG. 1(a)) and the 3×3 glucose colorimetric kit (Example 1) into which glucose was dropped (FIG. 1(b)).

1.2 ΔRGB and RMS Quantification

In order to sense and quantify the color change of the 3×3 glucose colorimetric kit produced in Example 1 according to the present invention and comprising arrays, each consisting of the glucose colorimetric sensor, 0.1 M glucose was dropped into the 3×3 glucose colorimetric kit (Example 1). Then, ΔRGB was calculated, which is the difference between the RGB coordinates of the 3×3 glucose colorimetric kit comprising arrays, each consisting of the initial glucose colorimetric sensor into which glucose was not dropped, and the RGB coordinates of the 3×3 glucose colorimetric kit comprising arrays, each consisting of the glucose colorimetric sensor into which glucose was dropped. From the ΔRGB, RMS was calculated, and the results are shown in Table 4 below.

TABLE 4

| 1 | | | | 2 | | | | 3 | | | |
|---|---|---|---|---|---|---|---|---|---|---|---|
| ΔR | ΔG | ΔB | RMS | ΔR | ΔG | ΔB | RMS | ΔR | ΔG | ΔB | RMS |
| 142 | 116 | 155 | 138.6 | 122 | 98 | 120 | 113.9 | 104 | 63 | 93 | 88.4 |
| 4 | | | | 5 | | | | 6 | | | |
| ΔR | ΔG | ΔB | RMS | ΔR | ΔG | ΔB | RMS | ΔR | ΔG | ΔB | RMS |
| 68 | 50 | 108 | 79.1 | 78 | 153 | 159 | 135.1 | 78 | 50 | 75 | 65.8 |
| 7 | | | | 8 | | | | 9 | | | |
| ΔR | ΔG | ΔB | RMS | ΔR | ΔG | ΔB | RMS | ΔR | ΔG | ΔB | RMS |
| 28 | 26 | 66 | 44.0 | 24 | 4 | 63 | 39.0 | 84 | 84 | [symbol] | 92.7 |

※RMS (root mean square) is calculated as the root mean square of ΔR² + ΔG² + ΔB². [symbol]
indicates data missing or illegible when filed

Referring to Table 4 above, it can be confirmed that the absolute values of ΔR of all the arrays were 20 or more and the absolute values of AB of all the arrays were 50 or more. In addition, the root mean square (RMS) for ΔRGB, quantified based on the RGB coordinates of each array consisting of the colorimetric sensor that changed color by addition of glucose, and the initial RGB coordinates of each array consisting of the colorimetric biosensor to which glucose was not added, was from 39.0 (array 8) to 138.6 (array 1).

From the above results, it was demonstrated that the glucose colorimetric kit comprising arrays, each consisting of the glucose colorimetric sensor according to the present invention, responded to glucose with high sensitivity.

### Experimental Example 2. Lactate Sensing

2.1. Confirmation of Color Change

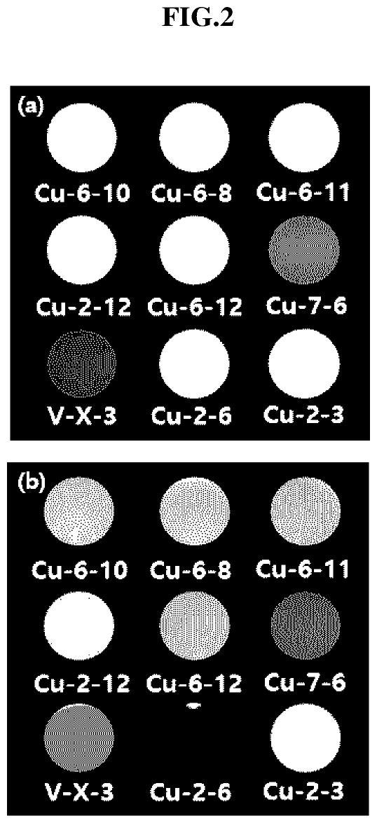

In order to sense and confirm the color change of the 3×3 lactate colorimetric kit produced in Example 2 according to the present invention and comprising arrays, each consisting of the lactate colorimetric sensor, 0.1 M lactate was dropped into the 3×3 lactate colorimetric kit (Example 2). The results are shown in FIG. 2.

Referring to FIG. 2, it could be confirmed that there was a clear difference between the 3×3 lactate colorimetric kit (Example 2) into which lactate was not dropped (FIG. 2(a)) and the 3×3 lactate colorimetric kit (Example 2) into which lactate was dropped (FIG. 2(b)).

2.2 ΔRGB and RMS Quantification

In order to sense and quantify the color change of the 3×3 lactate colorimetric kit produced in Example 2 according to the present invention and comprising arrays, each consisting of the lactate colorimetric sensor, 0.1 M glucose was dropped into the 3×3 lactate colorimetric kit (Example 2). Then, ΔRGB was calculated, which is the difference between the RGB coordinates of the 3×3 lactate colorimetric kit comprising arrays, each consisting of the initial lactate colorimetric sensor into which lactate was not dropped, and the RGB coordinates of the 3×3 lactate colorimetric kit comprising arrays, each consisting of the lactate colorimetric sensor into which lactate was dropped. From the ΔRGB, RMS was calculated, and the results are shown in Table 5 below.

TABLE 5

| 1 | | | | 2 | | | | 3 | | | |
|---|---|---|---|---|---|---|---|---|---|---|---|
| ΔR | ΔG | ΔB | RMS | ΔR | ΔG | ΔB | RMS | ΔR | ΔG | ΔB | RMS |
| 75 | 4 | 206 | [symbol] | 72 | 6 | 202 | 123.9 | 72 | 9 | 162 | 102.5 |
| 4 | | | | 5 | | | | 6 | | | |
| ΔR | ΔG | ΔB | RMS | ΔR | ΔG | ΔB | RMS | ΔR | ΔG | ΔB | RMS |
| 66 | 6 | 5 | 38.4 | 65 | 6 | 186 | [symbol] | 78 | 11 | 18 | 46.7 |
| 7 | | | | 8 | | | | 9 | | | |
| ΔR | ΔG | ΔB | RMS | ΔR | ΔG | ΔB | RMS | ΔR | ΔG | ΔB | RMS |
| 30 | 37 | 144 | [symbol] | 131 | 138 | 255 | 183.7 | 3 | 20 | 5 | 25.5 |

※RMS (root mean square) is calculated as the root mean square of ΔR² + ΔG² + ΔB². [symbol]
indicates data missing or illegible when filed

Referring to Table 5 above, it can be confirmed that the absolute values of ΔR of all the arrays were 20 or more. In addition, the root mean square (RMS) for ΔRGB, quantified based on the RGB coordinates of each array consisting of the colorimetric sensor that changed color by addition of lactate, and the initial RGB coordinates of each array consisting of the colorimetric biosensor to which lactate was not added, was from 25.5 (array 9) to 126.6 (array 1).

From the above results, it was demonstrated that the lactate colorimetric kit comprising arrays, each consisting of the lactate colorimetric sensor according to the present invention, responded to lactate with high sensitivity.

### Experimental Example 3. Pyruvate Sensing

3.1. Confirmation of Color Change

In order to sense and confirm the color change of the 3×3 pyruvate colorimetric kit produced in Example 3 according to the present invention and comprising arrays, each consisting of the pyruvate colorimetric sensor, 0.1 M glucose was dropped into the 3×3 pyruvate colorimetric kit (Example 3). The results are shown in FIG. 1.

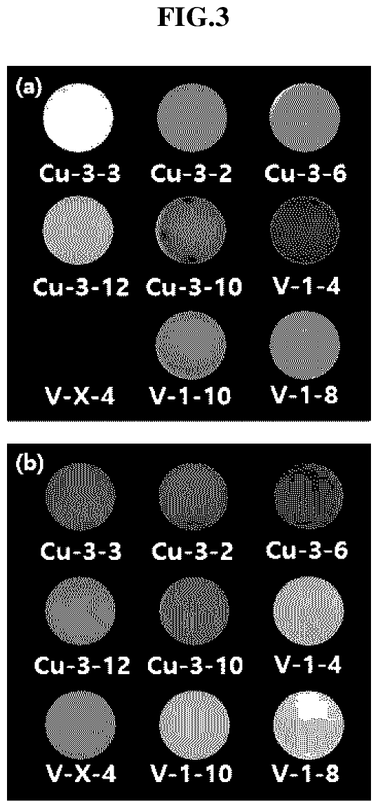

Referring to FIG. 3, it could be confirmed that there was a clear difference between the 3×3 pyruvate colorimetric kit (Example 3) into which pyruvate was not dropped (FIG. 3(a)) and the 3×3 pyruvate colorimetric kit (Example 3) into which glucose was dropped (FIG. 3(b)).

3.2 ΔRGB and RMS Quantification

In order to sense and quantify the color change of the 3×3 pyruvate colorimetric kit produced in Example 3 according to the present invention and comprising arrays, each consisting of the pyruvate colorimetric sensor, 0.1 M glucose was dropped into the 3×3 pyruvate colorimetric kit (Example 3). Then, ΔRGB was calculated, which is the difference between the RGB coordinates of the 3×3 pyruvate colorimetric kit comprising arrays, each consisting of the initial pyruvate colorimetric sensor into which pyruvate was not dropped, and the RGB coordinates of the 3×3 pyruvate colorimetric kit comprising arrays, each consisting of the pyruvate colorimetric sensor into which pyruvate was dropped. From the ΔRGB, RMS was calculated, and the results are shown in Table 6 below.

TABLE 6

| 1 | | | | 2 | | | | 3 | | | |
|---|---|---|---|---|---|---|---|---|---|---|---|
| ΔR | ΔG | ΔB | RMS | ΔR | ΔG | ΔB | RMS | ΔR | ΔG | ΔB | RMS |
| 216 | 144 | 46 | 152.2 | 156 | 42 | 16 | 93.7 | 180 | 87 | 20 | 116.0 |
| 4 | | | | 5 | | | | 6 | | | |
| ΔR | ΔG | ΔB | RMS | ΔR | ΔG | ΔB | RMS | ΔR | ΔG | ΔB | RMS |
| 174 | 78 | 17 | 110.5 | 133 | 28 | 69 | 88.0 | 83 | 84 | 174 | 121.4 |
| 7 | | | | 8 | | | | 9 | | | |
| ΔR | ΔG | ΔB | RMS | ΔR | ΔG | ΔB | RMS | ΔR | ΔG | ΔB | RMS |
| 17 | 78 | 132 | 89.1 | 80 | 50 | 138 | 96.5 | 84 | 44 | 140 | 97.6 |

※RMS (root mean square) is calculated as the root mean square of ΔR² + ΔG² + ΔB².

Referring to Table 6 above, it can be confirmed that the absolute values of ΔR of all the arrays were 20 or more and the absolute values of AB of all the arrays were 15 or more while the absolute values of ΔG were 20. In addition, the root mean square (RMS) for ΔRGB, quantified based on the RGB coordinates of each array consisting of the colorimetric sensor that changed color by addition of pyruvate, and the initial RGB coordinates of each array consisting of the colorimetric biosensor to which pyruvate was not added, was from 88.0 (array 5) to 152.2 (array 1).

From the above results, it was demonstrated that the pyruvate colorimetric kit comprising arrays, each consisting of the pyruvate colorimetric sensor according to the present invention, responded to pyruvate with high sensitivity.

While the present invention has been described with reference to the illustrative embodiments, those skilled in the art to which the present invention pertains will appreciate that that the present invention may be embodied in other specific forms without departing from the technical spirit or essential characteristics of the present invention. Therefore, the embodiments described above are considered to be illustrative in all respects and not restrictive.

1. A colorimetric biosensor comprising a transition metal, a solvent and an additive.

2. The colorimetric biosensor of claim 1, further comprising M13 bacteriophage.

3. The colorimetric biosensor of claim 1, which is for sensing glucose, lactate or pyruvate.

4. The colorimetric biosensor of claim 1, wherein the transition metal is at least one selected from the group consisting of copper (Cu), iron (Fe), cobalt (Co), chromium (Cr), manganese (Mn), nickel (Ni) and vanadium (V).

5. The colorimetric biosensor of claim 1, wherein the solvent is at least one selected from the group consisting of water, acetone, methanol, ethanol, n-propanol, isopropanol, n-butanol, chloroform, dichloromethane (DCM), toluene, tetrahydrofuran (THF), pyridine, diethyl ether, ethyl acetate, nitromethane, acetonitrile, ammonia, N,N-dimethylformamide (DMF), dimethyl sulfoxide (DMSO), formic acid, and propylenecarbonate.

6. The colorimetric biosensor of claim 1, wherein the additive is at least one selected from the group consisting of a halogen, sodium fluoride (NaF), sodium cyanate (NaNCO), sulfuric acid (H₂SO₄), nitric acid (HNO₃), oxalic acid, hydrogen peroxide (H₂O₂), hydrogen cyanide (HCN), sodium hydroxide (NaOH), hydroxylamine, ethylene amine, 4-nitrophenol, dithionate (S₂O₆²⁻), diborane, sodium borohydride, iodine (I₂), tris-2 carboxyethyl phosphine hydrochloride (TCEP), and dimethylamine borane (DMAB).

7. A colorimetric biokit comprising a plurality of arrays, each consisting of a colorimetric biosensor comprising a transition metal, a solvent, and an additive.

8. The colorimetric biokit of claim 7, wherein colors of the arrays change depending on the presence or absence of an analyte.

9. The colorimetric biokit of claim 7, wherein the analyte is glucose, lactate, or pyruvate.

10. The colorimetric biokit of claim 9, wherein the analyte is glucose, and the colorimetric biokit has a configuration shown in Table 1 below:
TABLE 1

| 1 | 2 | 3 |
|---|---|---|
| Copper-acetone- | Copper-water- | Copper-ethanol- |
| hydroxylamine | hydroxylamine | hydroxylamine |
| 4 | 5 | 6 |
| Copper-propylene | Iron-isopropanol- | Copper-methanol- |
| carbonate- | hydroxylamine | hydroxylamine |
| hydroxylamine | | |
| 7 | 8 | 9 |
| Copper-DMSO- | Copper-DMF- | Cobalt-pyridine- |
| hydroxylamine | hydroxylamine | hydroxylamine. |

11. The colorimetric biokit of claim 10, wherein it is determined that glucose is present, when a root mean square (RMS) for ΔRGB, quantified based on RGB coordinates of each array consisting of the colorimetric biosensor that changed color by addition of glucose, and initial RGB coordinates of each array consisting of the colorimetric biosensor to which glucose has not been added, is 20 to 300.

12. The colorimetric biokit of claim 9, wherein the analyte is lactate, and the colorimetric biokit has a configuration shown in Table 2 below:
TABLE 2

| 1 | 2 | 3 |
|---|---|---|
| Copper-4-nitrophenol- | Copper-4-nitrophenol- | Copper-4-nitrophenol- |
| ethanol | isopropanol | methanol |
| 4 | 5 | 6 |
| Copper-DMAB-water | Copper-4-nitrophenol- | Copper-iodine- |
| | water | acetonitrile |
| 7 | 8 | 9 |
| Vanadium-halogen | Copper-DMAB- | Copper-DMAB-acetone. |
| (Cl)-acetone | acetonitrile | |

13. The colorimetric biokit of claim 12, wherein it is determined that lactate is present, when a root mean square (RMS) for ΔRGB, quantified based on RGB coordinates of each array consisting of the colorimetric biosensor that changed color by addition of lactate, and initial RGB coordinates of each array consisting of the colorimetric biosensor to which lactate has not been added, is 20 to 300.

14. The colorimetric biokit of claim 9, wherein the analyte is pyruvate, and the colorimetric biokit has a configuration shown in Table 3 below:
TABLE 3

| 1 | 2 | 3 |
|---|---|---|
| Copper- | Copper- | Copper- |
| hydroxylamine- | hydroxylamine- | hydroxylamine- |
| acetone | pyridine | acetonitrile |
| 4 | 5 | 6 |
| Copper- | Copper- | Vanadium-hydrogen |
| hydroxylamine- | hydroxylamine- | peroxide-DMF |
| water | ethanol | |
| 7 | 8 | 9 |
| Vanadium-halogen | Vanadium-hydrogen | Vanadium-hydrogen |
| (Cl)-DMF | peroxide-ethanol | peroxide-isopropanol. |

15. The colorimetric biokit of claim 14, wherein it is determined that pyruvate is present, when a root mean square (RMS) for ΔRGB, quantified based on RGB coordinates of each array consisting of the colorimetric biosensor that changed color by addition of pyruvate, and initial RGB coordinates of each array consisting of the colorimetric biosensor to which pyruvate has not been added, is 20 to 300.

16. A sensing method using a colorimetric biosensor comprising steps of:
(S1) dropping an analyte into a colorimetric biokit comprising a plurality of arrays; and
(S2) sensing changes in colors of the plurality of arrays having the analyte dropped thereinto.
